(19) United States
(12) Patent Application Publication
(10) Pub. No.: US 2020/0247930 A1
(43) Pub. Date: Aug. 6, 2020

(54) DMA-CO-TEMPO POLYMER FOR THERAPEUTIC ROS SCAVENGING

(71) Applicant: Vanderbilt University, Nashville, TN (US)

(72) Inventors: Craig L. Duvall, Nashville, TN (US); Taylor Kavanaugh, Nashville, TN (US); Eric Dailing, Nashville, TN (US); Bryan Dollinger, Nashville, TN (US); Carlisle Dejulius, Nashville, TN (US)

(21) Appl. No.: 16/652,236

(22) PCT Filed: Oct. 1, 2018
(86) PCT No.: PCT/US2018/053787

§ 371 Date: Mar. 30, 2020

Related U.S. Application Data
(60) Provisional application No. 62/565,921, filed on Sep. 29, 2017.

(51) Int. Cl. C08F 265/10; A61K 31/787

(52) U.S. Cl. CPC C08F 265/10; A61K 31/787

(57) ABSTRACT

Provided herein are a reactive oxygen species (ROS) scavenging composition, a method of forming a ROS scavenging composition, and a method of treating a subject having a (ROS) perpetuated disease. The ROS scavenging composition includes a co-polymer of a hydrophilic monomer and a grafting monomer, and a ROS scavenging compound grafted to the grafting monomer. The method of forming a ROS scavenging composition includes forming a co-polymer of a hydrophilic monomer and a grafting monomer, and grafting ROS scavenging compound onto the co-polymer. The method of treating a subject having a ROS perpetuated disease includes administering a therapeutically effective amount of an ROS scavenging composition comprising a co-polymer of a hydrophilic monomer and a grafting monomer, and a ROS scavenging compound grafted to the grafting monomer, to the subject in need thereof.

### RELATED APPLICATIONS

This application claims the benefit of U.S. Provisional Application Ser. No. 62/565,921, filed Sep. 29, 2017, the entire disclosure of which is incorporated herein by this reference.

### GOVERNMENT INTEREST

This invention was made with government support under grant number OR130302 awarded by the Department of Defense (DOD), and grant number 1445197 awarded by the National Science Foundation (NSF). The government has certain rights in the invention.

### TECHNICAL FIELD

The present invention relates to methods and articles for reactive oxygen species (ROS) scavenging. In particular, the presently-disclosed subject matter relates to copolymers for ROS scavenging and methods of forming such copolymers.

### BACKGROUND

Elevated reactive oxygen species (ROS) or “oxidative stress” is a primary driver of DNA damage, protein denaturation, lipid peroxidation, and cell death associated with chronic inflammatory diseases such as osteoarthritis (OA) and many others. Systemically-delivered OA therapies are hindered by the lack of vasculature within synovial joints where the pathology is presented. Local administration is practical for human OA treatment, but the therapeutic needs to be highly potent and long-lasting, as small volumes can typically only be injected every few months.

One potential therapeutic includes Tempol (4-hydroxy-Tempo), a small molecule superoxide dismutase memetic that catalyzes the disproportionation of superoxide to hydrogen peroxide and water. Although hydroxy-tempo is therapeutically promising, it is a very small, water soluble molecule that has poor retention time when delivered locally. As such, for local delivery applications, the small molecule Tempol is cleared out very rapidly. In an attempt to address this rapid clearance, polymeric tempo (poly(tempo)) has been formed where tempo molecules are grafted at high density (every monomer repeat) within a block of the polymer (FIG. 1). Although this high density polymer Tempol is an effective way to alter Tempol pharmacokinetics, such polymers have not provided optimal or highly potent ROS disproportionation because homopolymer poly(tempo) is excessively hydrophobic, making bioavailability low in aqueous biologic environments.

Accordingly, there remains a need for articles and methods to effectively deliver Tempol in a form that can be retained better than single small molecule form but maintain potent ability to disproportionate ROS.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes, in some embodiments, a reactive oxygen species (ROS) scavenging composition comprising a co-polymer of a hydrophilic monomer and a grafting monomer, and a ROS scavenging compound grafted to the grafting monomer. In some embodiments, the hydrophilic monomer includes dimethylacrylamide (DMA), oligo(ethyleneglycol) acrylate (OEGA), oligo(ethyleneglycol) methacrylate (OEGMA), zwitterionic monomers, or combinations thereof. In some embodiments, the ROS scavenging compound comprises an amine containing ROS scavenging compound. In one embodiment, the amine containing ROS scavenging compound includes 4-amino-Tempo, boronic esters, 2-(Methylthio)ethylamine, 2-(Ethylthio)ethylamine, or combinations thereof. In some embodiments, the grafting monomer comprises a portion of an amine reactive monomer. In one embodiment, the amine reactive monomer is selected from the group consisting of pentafluorophenyl acrylate (PFPA), N-hydroxysuccinimide containing monomers, and combinations thereof. In another embodiment, the amine reactive monomer is PFPA and the grafting monomer comprises the portion of the PFPA remaining after pentafluorophenol is replaced by the amine containing ROS scavenging compound. In some embodiments, the ROS scavenging compound comprises a hydroxy containing ROS scavenging compound. In one embodiment, the hydroxy containing ROS scavenging compound comprises 4-hydroxy-Tempo.

In some embodiments, the composition comprises a [hydrophilic monomer]:[ROS scavenging compound] ratio of between 1:99 and 99:1. In some embodiments, the [hydrophilic monomer]:[ROS scavenging compound] ratio is between 10:90 and 95:5. In one embodiment, the hydrophilic monomer comprises DMA, the ROS scavenging compound comprises Tempo, and the DMA:Tempo ratio is between 60:40 and 80:20. In one embodiment, the hydrophilic monomer comprises OEGA/OEGMA, the ROS scavenging compound comprises Tempo, and the OEGA/OEGMA:Tempo ratio is at least 10:90.

Also provided herein, in some embodiments, is method of forming a reactive oxygen species (ROS) scavenging composition comprising forming a co-polymer of a hydrophilic monomer and a grafting monomer, and grafting ROS scavenging compound onto the co-polymer. In some embodiments, the hydrophilic monomer is selected from the group consisting of dimethylacrylamide (DMA), oligo(ethyleneglycol) acrylate (OEGA), oligo(ethyleneglycol) methacrylate (OEGMA), zwitterionic monomers, and combinations thereof. In some embodiments, the grafting monomer is selected from the group consisting of a hydroxy reactive monomer, pentafluorophenyl acrylate (PFPA), N-hydroxysuccinimide containing monomers, and combinations thereof. In some embodiments, the ROS scavenging compound is selected from the group consisting of 4-amino-Tempo, 4-hydroxy-Tempo, boronic esters, 2-(Methylthio)ethylamine, 2-(Ethylthio)ethylamine, and combinations thereof. In one embodiment, the grafting monomer comprises pentafluorophenyl acrylate (PFPA), the ROS scavenging compound comprises 4-amino-Tempo, and grafting the 4-amino-Tempo to the co-polymer comprises replacing a pentafluorophenol group of the PFPA with the 4-amino-Tempo. In some embodiments, the forming of the co-polymer comprises reversible addition-fragmentation chain-transfer (RAFT) polymerization.

Further provided herein, in some embodiments, is a method of treating a subject having a reactive oxygen species (ROS) perpetuated disease comprising administering a therapeutically effective amount of an ROS scavenging composition comprising a co-polymer of a hydrophilic monomer and a grafting monomer, and a ROS scavenging compound grafted to the grafting monomer.

### BRIEF DESCRIPTION OF THE DRAWINGS

The novel features of the subject matter of the present disclosure are set forth with particularity in the appended claims. A better understanding of the features and advantages of the presently disclosed subject matter will be obtained by reference to the following detailed description that sets forth illustrative embodiments, in which the principles of the present disclosure are used, and the accompanying drawings of which:

FIG. 1 shows a schematic view illustrating synthesis of poly(Tempo).

FIG. 2 shows an image illustrating the structure of DMA-co-PFPA.

FIG. 3 shows an image illustrating the structure of DMA-co-Tempo.

FIG. 4 shows a graph illustrating the bioavailability of dimethylacrylamide (DMA)-co-Tempo.

FIGS. 5A-B show a graph and image illustrating bioavailability of polyTempo as compared to iron, Tempol, and a control. (A) Shows an image illustrating a schematic representation of Ferric Reducing Antioxidant Power (FRAP), an antioxidant assay performed in water. (B) Shows a graph illustrating the performance of iron, 4-hydroxy-Tempo, and polyTempo (DMA:Tempo 0:100) as determined in the FRAP assay.

FIGS. 6A-B show graphs illustrating the absorbance of DMA-co-Tempo polymers with various DMA:Tempo ratios, as determined by FRAP assay, which indicates the reducing (antioxidant) potential of the polymers. (A) shows a graph illustrating the absorbance of DMA-co-Tempo polymers at concentration of 1 mg/mL with various DMA:Tempo ratios. (B) shows a graph illustrating the absorbance of DMA-co-Tempo polymers at concentration of 5 mM Tempo with various DMA:Tempo ratios.

FIG. 7 shows a graph illustrating superoxide scavenging of 60:40 DMA:Tempo, as compared to no treatment, in a xanthine/xanthine oxidase system.

FIG. 8 shows a schematic illustrating synthesis of dimethylacrylamide (DMA)-co-Tempo. X (DMA): 50-90%; Y (Tempo): 10-50%.

FIG. 9 shows a graph illustrating 19F NMR of DMA:Tempo 60:40.

FIGS. 10A-B show a graph and a table illustrating the changes in molecular weight of DMA-co-Tempo as the percentage of Tempo is varied. (A) Shows a graph illustrating refractive index versus time of DMA-co-Tempo polymers having DMA:Tempo ratios of 50:50, 60:40, 70:30, 80:20, 90:10, and 100:0. (B) Shows a table listing the predicted molecular weight and polydispersity of the polymers in A.

FIGS. 11A-B show graphs illustrating the amount of Tempo present in each polymer after grafting, as quantified through electron spin resonance (ESR). (A) Shows a graph illustrating intensity versus [G] for DMA-co-Tempo 60:40. (B) Shows a graph illustrating relative Tempo content versus predicted percent Tempo of the polymers in FIG. 10A.

FIG. 12 shows a graph illustrating the concentration of reactive oxygen species (ROS) in air pouch exudate at DMA-co-Tempo (60:40) concentrations of 0 mg/mL, 0.01 mg/mL, 0.1 mg/mL, and 1 mg/mL.

FIG. 13 shows a graph illustrating the distribution of cell phenotype and total number of cells in air pouch exudate at DMA-co-Tempo (60:40) concentrations of 0 mg/mL, 0.01 mg/mL, 0.1 mg/mL, and 1 mg/mL.

FIG. 14 shows a graph illustrating reduction in concentration of ROS in air pouch exudate by DMA-co-Tempo polymers.

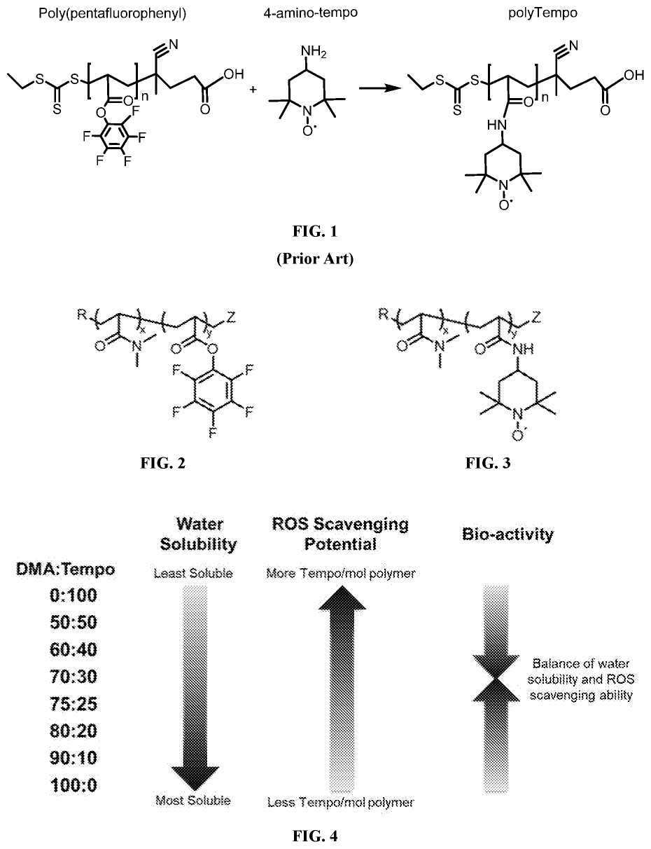

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims, unless the context clearly dictates otherwise. Thus, for example, reference to “a polypeptide” includes one or more of such polypeptides, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Provided herein are reactive oxygen species (ROS) scavenging compositions, methods of forming ROS scavenging compositions, and methods of using ROS scavenging compositions. In some embodiments, the ROS scavenging compositions include any suitable ROS scavenging compound grafted onto a co-polymer of a grafting monomer and a hydrophilic monomer. In one embodiment, the ROS scavenging compound includes a superoxide dismutase (SOD) that catalyzes the disproportionation of superoxide to hydrogen peroxide and water. In another embodiment, the SOD includes, but is not limited to, a small molecule SOD, a SOD mimetic, any other suitable SOD, or a combination thereof. For example, one suitable SOD includes 4-hydroxy-Tempo (Tempol), a small molecule SOD mimetic. In such embodiments, the ROS scavenging composition includes at least one Tempol molecule grafted onto the co-polymer of the grafting monomer and the hydrophilic monomer. Other suitable SODs include, but are not limited to, boronic esters. Other suitable ROS scavenging compounds include, but are not limited to, 2-(Methylthio)ethylamine, 2-(Ethylthio)ethylamine, any other ROS scavenging compound that reacts with hydrogen peroxide and/or hypochlorite, or a combination thereof.

The grafting monomer includes any suitable monomer or combination of monomers for grafting the ROS scavenging compound(s) thereto. As will be appreciated by those skilled in the art, the grafting monomer may differ depending upon the specific ROS scavenging compound selected. For example, in one embodiment, the ROS scavenging compound includes Tempol, or any other suitable ROS scavenging compound including a hydroxy group, and the grafting monomer includes a hydroxy reactive monomer. In another embodiment, the ROS scavenging compound includes amine modified Tempo (4-amino-Tempo), or any other suitable ROS scavenging compound including an amine group, and the grafting monomer includes an amine reactive monomer. Suitable amine reactive monomers include, but not limited to, pentafluorophenyl acrylate (PFPA), N-hydroxysuccinimide containing monomers (e.g., acrylic acid N-hydroxysuccinimide ester, methacrylic acid N-hydroxysuccinimide ester), any other monomers capable of grafting the amine containing ROS scavenging compound thereto, or a combination thereof. Although described above with regard to hydroxy and amine reactive grafting monomers, as will be appreciated by those skilled in the art, the disclosure is not so limited and may include any other suitable monomer for grafting the ROS scavenging compound thereto.

The hydrophilic monomer includes any monomer suitable for copolymerizing with the grafting monomer and offsetting the hydrophobicity of the ROS scavenging compound. Suitable hydrophilic monomers include, but are not limited to, dimethylacrylamide (DMA), oligo(ethyleneglycol) acrylate (OEGA), oligo(ethyleneglycol) methacrylate (OEGMA), zwitterionic monomers, any other hydrophilic monomer suitable for offsetting the hydrophobicity of the ROS scavenging compound, or a combination thereof. For example, in one embodiment, the grafting monomer includes an amine reactive monomer, and the co-polymer of the amine reactive monomer and the hydrophilic monomer includes DMA-co-PFPA (FIG. 2). In another embodiment, the amine modified Tempo, 4-amino-Tempo, is grafted onto the co-polymer of DMA and PFPA to form the ROS scavenging composition poly(DMA-co-Tempo) (FIG. 3). As will be appreciated by those skilled in the art, upon grafting the ROS scavenging compound to the co-polymer a portion of the grafting monomer is replaced by the ROS scavenging compound. Accordingly, when referring to the ROS scavenging composition, the grafting monomer is the portion of the monomer remaining after the ROS scavenging compound is grafted thereto. In a further embodiment, DMA may be replaced with any other suitable hydrophilic monomer, PFPA may be replaced with any other suitable grafting monomer, and/or Tempo may be replaced with any other suitable ROS scavenging compound. For example, in one embodiment, the ROS scavenging composition includes OEGA and/or OEGMA in combination with PFPA and 4-amino-Tempo. In another embodiment, the ROS scavenging composition includes N-hydroxysuccinimide containing monomers in combination with any suitable hydrophilic monomers and an amine containing ROS scavenging compound.

Additionally or alternatively, in some embodiments, the ROS scavenging composition includes a combination of different ROS scavenging compounds, grafting monomers, and/or hydrophilic monomers. For example, in one embodiment, the ROS scavenging composition includes multiple amine containing or amine modified ROS scavenging compounds grafted onto a co-polymer of an amine reactive monomer and a hydrophilic monomer. In another embodiment, the ROS scavenging compounds include 4-amino-Tempo as well as 2-(Methylthio)ethylamine and/or 2-(Ethylthio)ethylamine, the amine reactive monomer includes PFPA, and the hydrophilic monomer includes PFPA. In a further embodiment, the 4-amino-Tempo grafted to the ROS scavenging composition reacts with superoxide, while the 2-(Methylthio)ethylamine and/or 2-(Ethylthio)ethylamine grafted to the ROS scavenging composition reacts with hydrogen peroxide and hypochlorite. In another example, the ROS scavenging composition may include more than one grafting monomer (e.g., multiple amine reactive monomers, amine reactive monomers and hydroxy reactive monomers, or other any other combination of grafting monomers) and/or more than one hydrophilic monomer (e.g., multiple hydrophilic monomers of different sizes).

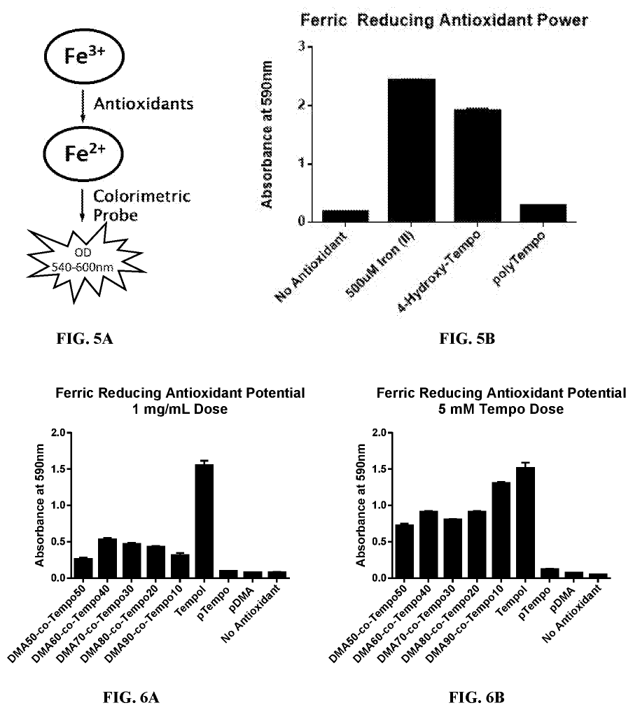

Co-polymers having a higher ratio and/or concentration (mol %) of DMA are more hydrophilic than co-polymers having a comparatively lower ratio and/or concentration of DMA, while co-polymers having a comparatively higher ratio and/or concentration of Tempo are expected to have increased ROS scavenging potential (FIG. 4). However, in contrast to the expected increase in ROS scavenging due to the higher density of the antioxidant entity tempo, 100% polyTempo (i.e., a DMA:Tempo ratio of 0:100) performed poorly in antioxidant assays such as the ferric reducing antioxidant power (FRAP) assay performed in water (FIG. 5A). In fact, as illustrated in FIG. 5B, 100% polyTempo performed significantly worse (i.e., had significantly decreased reducing activity) than 4-hydroxy-Tempo, the free drug, at the same tempo content. Without wishing to be bound by theory, it is believed that in these 100% polyTempo polymers, the hydrophobic poly(Tempo) is not the molecularly solvated and may be aggregated, burying the antioxidant therein and thus decreasing the bioavailability of Tempo.

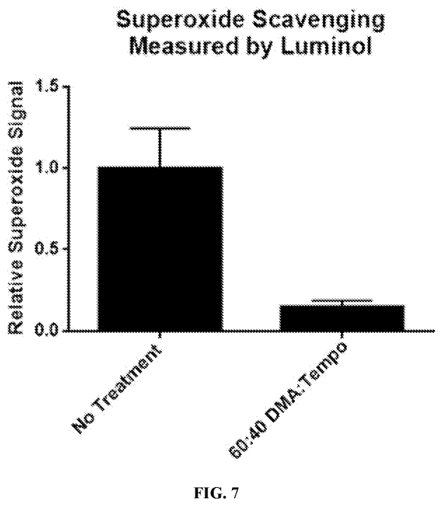

Although counter-intuitive, the instant inventors have discovered that decreasing ROS scavenging compound content/density in the polymer by copolymerizing with a hydrophilic monomer actually increases the antioxidant function of the product. More specifically, referring to FIGS. 6A-B, as compared to 4-amino-Tempo grafted directly to poly(PFPA) (i.e., 100% poly(Tempo) or DMA:Tempo 0:100), which is hydrophobic and not water soluble, the instant DMA-co-Tempo compositions have increased water solubility, improved bioavailability, and stronger antioxidant function. Additionally, as illustrated in FIG. 7, these compositions provide effective superoxide scavenging.

Accordingly, in some embodiments, the [hydrophilic]:[scavenger] ratio and/or concentration is selected and/or adjusted to provide and/or modify the hydrophilicity/ROS scavenging/antioxidant potential and thus the bio-activity of the composition. Suitable [hydrophilic]:[scavenger] ratios include, but are not limited to, between about 1:99 and about 99:1, about 5:95 and about 95:5, between about 10:90 and about 95:5, between about 20:80 and about 95:5, between about 30:70 and about 95:5, between about 40:60 and about 95:5, between about 50:50 and about 95:5, between about 55:45 and about 95:5, between about 60:40 and about 95:5, between about 60:40 and about 90:10, or any combination, sub-combination, range, or sub-range thereof. In some embodiments, the ratio is selected based upon the size of the hydrophilic monomer. For example, in one embodiment, the composition includes a comparatively larger amount of smaller hydrophilic monomers, such as DMA, or a comparatively smaller amount of larger hydrophilic monomers, such as OEGA/OEGMA. In another embodiment, the comparatively smaller amount of larger hydrophilic monomers permits a higher density of scavenger and/or a higher activity.

In some embodiments, the ROS scavenging composition includes DMA-co-Tempo with a [DMA]: [Tempo] ratio of between about 1:99 and about 99:1, about 5:95 and about 95:5, between about 10:90 and about 95:5, between about 20:80 and about 95:5, between about 30:70 and about 95:5, between about 40:60 and about 95:5, between about 50:50 and about 95:5, between about 55:45 and about 95:5, between about 60:40 and about 95:5, between about 60:40 and about 90:10, or any combination, sub-combination, range, or sub-range thereof. In one embodiment, the [DMA]: [Tempo] ratio includes a DMA concentration of at least 50. In another embodiment, the [DMA]: [Tempo] ratio is at least 60:40, including, for example, ratios of 60:40, 65:35, 70:30, 75:25, 80:20, 85:15, 90:10, or any combination, sub-combination, range, or sub-range thereof. In a further embodiment, the [DMA]:[Tempo] ratio is between 60:40 and 80:20, 60:40 and 75:25, 60:40 and 70:30, or any combination, sub-combination, range, or sub-range thereof. In some embodiments, the ROS scavenging composition includes OEGA/OEGMA-co-Tempo with a [OEGA/OEGMA]:[Tempo] ratio of at least 10:90, including, for example, ratios of 10:90, 20:80, 25:75, 30:70, 35:65, 40:60, 45:55, 50:50, 55:45, 60:40, 65:35, 70:30, 75:25, 80:20, 85:15, 90:10, or any combination, sub-combination, range, or sub-range thereof. In one embodiment, the [OEGA/OEGMA]:[Tempo] ratio is at least 20:80. In another embodiment, the [OEGA/OEGMA]: [Tempo] ratio is at least 70:30.

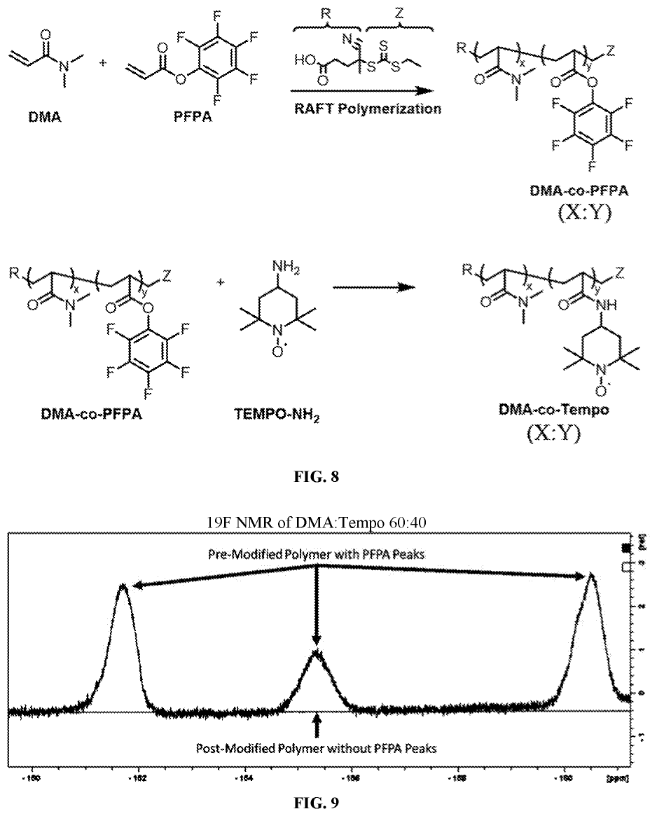

Also provided herein, in some embodiments, is a method of forming the ROS scavenging composition. In some embodiments, the method includes forming a co-polymer of a grafting monomer and a hydrophilic monomer using reversible addition-fragmentation chain-transfer (RAFT) polymerization. Next, the ROS scavenging compound is grafted onto the co-polymer. For example, in one embodiment, the method includes forming the co-polymer of an amine reactive monomer and a small hydrophilic monomer using RAFT polymerization, then grafting the amine modified small molecule superoxide dismutase mimetic onto the co-polymer. In another embodiment, a poly(DMA-co-Tempo) co-polymer is formed using RAFT polymerization, and then the amine modified Tempo (4-amino-Tempo) derivative is grafted onto the DMA-co-PFPA polymer (FIG. 8).

Further provided herein, in some embodiments, is a method of using the ROS scavenging composition. In some embodiments, the method includes treating a subject having and/or at risk of having an ROS perpetuated disease. In some embodiments, the method includes administering a therapeutically effective amount of the ROS scavenging composition to a subject in need thereof. In one embodiment, the subject includes any subject having and/or at risk of having an ROS perpetuated disease, such as, but not limited to, inflammation, a chronic inflammatory disease, chronic wounds, arthritis, osteoarthritis, inflammatory bowel disease, ischemia reperfusion injury in the brain, heart, or other tissues, any other disease where treatment involves ROS scavenging, or a combination thereof. In another embodiment, the subject includes any subject that would benefit from antioxidant activity. As used herein, the term “subject” refers to any mammal, including, but not limited to, a human.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

This example involves co-polymerizing PFPA and DMA at varying ratios then grafting on 4-Amino-Tempo to make a more hydrophilic polymeric form of Tempo (FIG. 8). The resulting polymers were investigated to determine their antioxidant potential, which confirmed increasing water solubility was able to increase antioxidant potential compared to 100% poly(Tempo) even though the relative content of the antioxidant molecule was lower. DMA-co-Tempo at ratios of 60:40, 70:30, 75:25, and 80:20 were especially promising for optimizing water solubility and antioxidant potential (FIG. 6). Additionally, the superoxide scavenging of 60:40 DMA:Tempo polymer in a xanthine/xanthine oxidase system was measured by superoxide activation of luminol (FIG. 7).

### Example 2

Materials and Methods

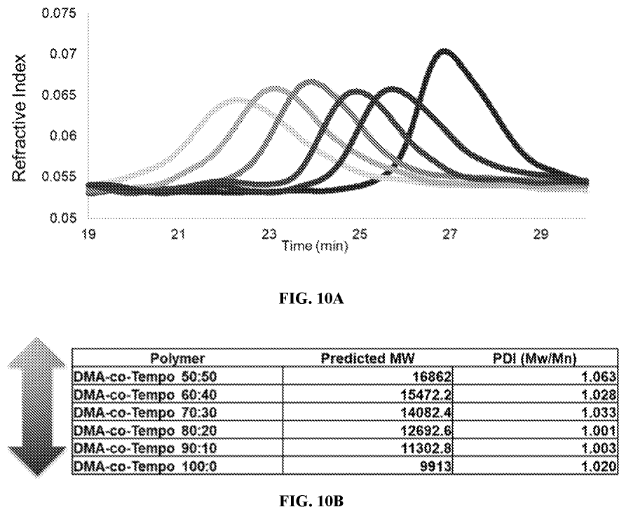

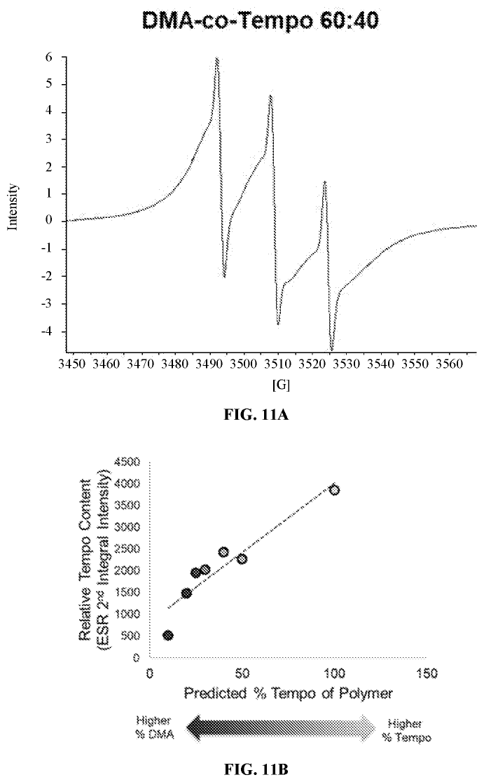

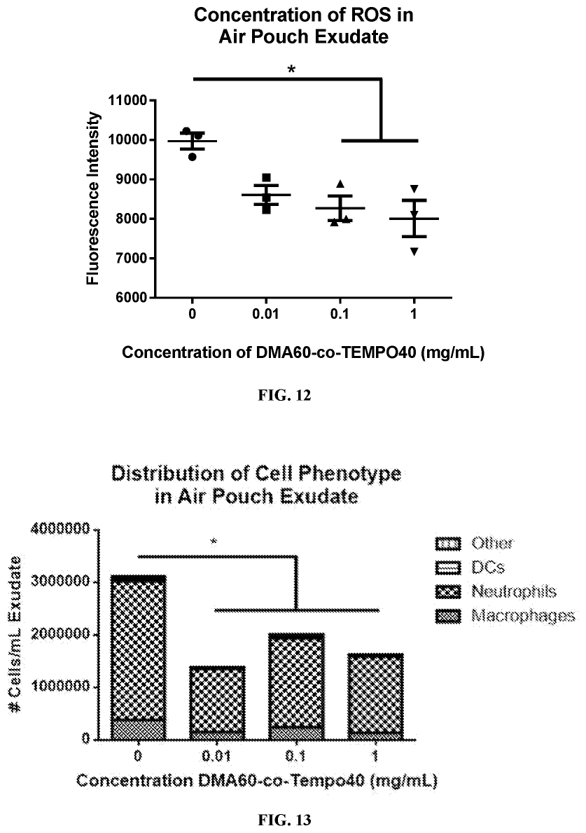

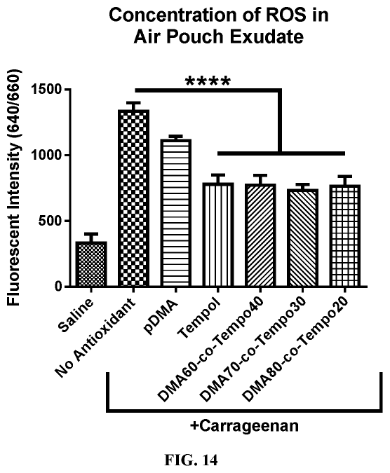

Reversible addition-fragmentation chain transfer (RAFT) polymerization was utilized to synthesize a co-polymer of dimethylacrylamide (DMA, small hydrophilic monomer) and pentafluorophenyl acrylate (PFPA), an amine-reactive monomer that was used to substitute in 4-amino-Tempo. DMA and PFPA were RAFT polymerized at varying molar ratios (50:50 to 90:10) with a target degree of polymerization of 100 units (FIG. 8). Polymers were characterized by nuclear magnetic resonance (NMR) spectroscopy (FIG. 9) and gel permeation chromatography (GPC) (FIGS. 10A-B) to confirm synthesis and post-polymerization modification. Electron spin resonance (ESR), a method that uses magnetic fields to detect free radicals or unpaired electrons, was also used to quantify the amount of Tempo present in each polymer after grafting (FIGS. 11A-B). Ferric reducing antioxidant potential and cytotoxicity were screened in vitro, and an air pouch inflammation model was used to test for in vivo antioxidant and anti-inflammatory properties. For the air pouch model, the lead DMA-co-Tempo polymer identified by in vitro screens (60:40 ratio) was co-injected with carrageenan (an inflammation inducing substrate), and ROS measurements (FIG. 12) and flow cytometry immune phenotyping (FIG. 13) were completed. Additionally, ROS measurements were compared to other polymers in the air pouch model (FIG. 14).

Results and Discussion

Successful grafting of tempo through replacement of pentafluorophenol by 4-amino-Tempo was confirmed by the disappearance of fluorine groups in the fluorine NMR spectrum of DMA-co-Tempo 60:40 (FIG. 9). DMA is a much smaller monomer than tempo, therefore polymers with higher percentages of DMA are predicted to be smaller and should elute latest. Increases in DMA-co-Tempo molecular weight with increases in percent Tempo were confirmed using GPC, while all of the polymers eluted in the appropriate order and had relatively small polydispersity (FIGS. 10A-B). Additionally, increased Tempo content was confirmed by ESR, with a linear trend correlating predicted percent tempo to the ESR spectrum intensity (FIGS. 11A-B).

No cytotoxicity was detectable among this polymer library (not shown). A ratio of 60:40 DMA:Tempo in the polymer backbone showed optimal antioxidant potential by FRAP assay (FIG. 6). Introduction of the 60 mol % DMA/40 mol % Tempo polymer into the air pouch led to a dose-dependent reduction in ROS as measured by reagents for measurement of reactive oxygen species levels (e.g., ROSStar®) (FIG. 12). Significant reduction in: total cell number along with total macrophages and neutrophils (FIG. 13), and pro-inflammatory cytokine IL1ß in exudate (not shown) was also observed for air pouch excipients that received DMA-co-Tempo. This shows a reduction in infiltration of neutrophils in vivo to a site of induced/extreme inflammation. Since neutrophils and their oxidative burst create oxidative stress and host tissue damage in inflammatory disease settings, the reduction in neutrophil infiltration indicates a reduction in oxidative stress and/or host tissue damage in such settings. Together, these data suggest that local inflammation can be controlled using this polymer treatment.

### CONCLUSION

Structurally-optimized DMA-co-Tempo polymers are promising for reduction of inflammation mediated by ROS.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Henrotin, Y, et al., Osteoarthritis and Cartilage, 2005. 13(8): p. 643-654.
2. Brownlee, M, Nature, 2001. 414(6865): p. 813-820.
3. Siwik, D A, et al., American Journal of Physiology-Cell Physiology, 2001. 280(1): p. C53-C60.
4. Larsen, C, et al., Journal of Pharmaceutical Sciences, 2008. 97(11): p. 4622-4654.
5. Vong, L B, et al., Gastroenterology, 2012. 143(4): p. 1027-+.
6. Wollberg, A R, Inflammation Research, 2005. 54: p. S169-S169.

It will be understood that various details of the presently disclosed subject matter can be changed without departing from the scope of the subject matter disclosed herein. Furthermore, the foregoing description is for the purpose of illustration only, and not for the purpose of limitation.

1. A reactive oxygen species (ROS) scavenging composition comprising:
a co-polymer of:
a hydrophilic monomer; and
a grafting monomer; and
a ROS scavenging compound grafted to the grafting monomer.

2. The composition of claim 1, wherein the hydrophilic monomer is selected from the group consisting of dimethylacrylamide (DMA), oligo(ethyleneglycol) acrylate (OEGA), oligo(ethyleneglycol) methacrylate (OEGMA), zwitterionic monomers, and combinations thereof.

3. The composition of claim 1, wherein the ROS scavenging compound comprises an amine containing ROS scavenging compound.

4. The composition of claim 3, wherein the amine containing ROS scavenging compound is selected from the group consisting of 4-amino-Tempo, boronic esters, 2-(Methylthio)ethylamine, 2-(Ethylthio)ethylamine, and combinations thereof.

5. The composition of claim 3, wherein the grafting monomer comprises a portion of an amine reactive monomer.

6. The composition of claim 5, wherein the amine reactive monomer is selected from the group consisting of pentafluorophenyl acrylate (PFPA), N-hydroxysuccinimide containing monomers, and combinations thereof.

7. The composition of claim 6, wherein the amine reactive monomer is PFPA and the grafting monomer comprises the portion of the PFPA remaining after pentafluorophenol is replaced by the amine containing ROS scavenging compound.

8. The composition of claim 1, wherein the ROS scavenging compound comprises a hydroxy containing ROS scavenging compound.

9. The composition of claim 8, wherein the hydroxy containing ROS scavenging compound comprises 4-hydroxy-Tempo.

10. The composition of claim 1, wherein the composition comprises a [hydrophilic monomer]:[ROS scavenging compound] ratio of between 1:99 and 99:1.

11. The composition of claim 10, wherein the [hydrophilic monomer]:[ROS scavenging compound] ratio is between 10:90 and 95:5.

12. The composition of claim 11, wherein the hydrophilic monomer comprises DMA, the ROS scavenging compound comprises Tempo, and the DMA:Tempo ratio is between 60:40 and 80:20.

13. The composition of claim 11, wherein the hydrophilic monomer comprises OEGA/OEGMA, the ROS scavenging compound comprises Tempo, and the OEGA/OEGMA:Tempo ratio is at least 10:90.

14. A method of forming a reactive oxygen species (ROS) scavenging composition comprising:
forming a co-polymer of a hydrophilic monomer and grafting monomer; and
grafting a ROS scavenging compound onto the co-polymer.

15. The method of claim 14, wherein the hydrophilic monomer is selected from the group consisting of dimethylacrylamide (DMA), oligo(ethyleneglycol) acrylate (OEGA), oligo(ethyleneglycol) methacrylate (OEGMA), zwitterionic monomers, and combinations thereof.

16. The method of claim 14, wherein the grafting monomer is selected from the group consisting of a hydroxy reactive monomer, pentafluorophenyl acrylate (PFPA), N-hydroxysuccinimide containing monomers, and combinations thereof.

17. The method of claim 14, wherein the ROS scavenging compound is selected from the group consisting of 4-amino-Tempo, 4-hydroxy-Tempo, boronic esters, 2-(Methylthio)ethyl amine, 2-(Ethylthio)ethylamine, and combinations thereof.

18. The method of claim 14, wherein the grafting monomer comprises pentafluorophenyl acrylate (PFPA), the ROS scavenging compound comprises 4-amino-Tempo, and grafting the 4-amino-Tempo to the co-polymer comprises replacing a pentafluorophenol group of the PFPA with the 4-amino-Tempo.

19. The method of claim 14, wherein the forming of the co-polymer comprises reversible addition-fragmentation chain-transfer (RAFT) polymerization.

20. A method of treating a subject having a reactive oxygen species (ROS) perpetuated disease comprising:
administering a therapeutically effective amount of the ROS scavenging composition of claim 1 to the subject in need thereof.
